(19) United States
(12) Patent Application Publication
(10) Pub. No.: US 2020/0108019 A1
(43) Pub. Date: Apr. 9, 2020

(54) Systems and Methods of Combining Cannabinoids with Sugar-Based Solution and of Creating Orally Dissolvable Tablets

(71) Applicants: Francesco Capaldi, Wakefield, MA (US); William Cumming, Bath, ME (US)

(72) Inventors: Francesco Capaldi, Wakefield, MA (US); William Cumming, Bath, ME (US)

(21) Appl. No.: 16/593,992

(22) Filed: Oct. 5, 2019

Related U.S. Application Data
(60) Provisional application No. 62/872,050, filed on Jul. 9, 2019.
(60) Provisional application No. 62/742,321, filed on Oct. 6, 2018.

(51) Int. Cl. A61K 9/20; A61K 31/353; A61K 36/77

(52) U.S. Cl. CPC A61K 9/2095; A61K 9/2009; A61K 36/77; A61K 9/2018; A61K 31/353; A61K 9/2013

(57) ABSTRACT

Systems, apparatus, and methods for combining a sugar-based solution, such as maple syrup, and cannabinoids are provided, in which a method, for instance, may comprise creating maple syrup and separately warming the maple syrup and a cannabinoid concentrate to a first temperature and combining them to create a mixture. The method also may comprise applying a bottom heat source and/or a top radiant heat source to the mixture and increasing the mixture's temperature to a second temperature sufficient to initiate crystallization, while stirring at a first RPM set point. The method also comprises discontinuing, upon the mixture reaching full crystallization, the heating and stirring RPMs. A method for creating an orally dissolvable tablet is also provided, comprising taking the combined cannabinoids and maple syrup in powder form, mixing with a secondary formulation and an excipient mixture, and pressing the resulting mixture into tablet form.

### CROSS REFERENCE TO RELATED APPLICATION

This application is related to and claims the benefit of U.S. Provisional Patent Application Ser. No. 62/742,321, titled “Systems and Methods of Combining Cannabinoids with Maple Syrup,” filed on Oct. 6, 2018, and U.S. Provisional Patent Application Ser. No. 62/872,050, titled “Systems and Methods of Combining Cannabinoids with Maple Syrup and of Creating Orally Dissolvable Tablets,” filed on Jul. 9, 2019, each of which is incorporated by reference herein in its entirety for all purposes.

### BACKGROUND OF THE INVENTION

### Field of the Invention

The invention relates to systems, methods, and apparatus involving orally dissolvable tablets and involving formulations of cannabis extracts for oral consumption, including the field of formation of tablets of orally dissolvable formulations of cannabis extracts.

Embodiments of the present invention generally relate to systems and methods for combining cannabinoids with sugar-based solutions, sucrose solutions, fructose solutions, and the like including, but not limited to, maple syrup, and of creating orally dissolvable tablets. More specifically, the present invention relates to systems and methods for combining a sugar-based solution, for example, maple syrup, and a concentrate containing cannabinoids into a crystallized form. The crystallized form, or powder created therefrom, is then combined with other ingredients to create an orally dissolvable tablet.

### Description of Related Art

The related art includes, for instance, formulation of edible compositions having cannabis extracts, such as gummi bears, cookies, brownies, chocolates, condiments, ‘fortified’ butter, and assorted beverages. Edible forms of cannabis, including food products, lozenges, and capsules, can produce noticeable, long-lasting effects. Many edibles contain a significant amount of tetrahydrocannabinol (THC). THC-dominant edibles are consumed for recreational and medical purposes and can induce a wide range of effects including relaxation, euphoria, increased appetite, fatigue, and anxiety. Some edibles feature other cannabinoids predominantly, most commonly cannabidiol (CBD) with very little THC. Cannabidiol has been identified as having multiple favorable effects for some users, including possible homeopathic results. CBD products are frequently used for several issues associated with wellness, including sleep, relaxation, mood, skin care, and focus. CBD may be extracted from hemp, also called industrial hemp, which includes plant varieties that typically contain very little THC and refer to the non-intoxicating varieties of cannabis. Hemp-derived CBD products typically contain less than 0.3% THC, so they do not produce the high typically associated with marijuana. Marijuana includes the plant varieties that typically contain significant THC content, largely due to intentional breeding efforts over the years, and refers to the intoxicating varieties of cannabis. For some people, CBD cannabinoids act like an antidote for those who have consumed too much THC.

Due to the complex chemical structure of cannabis and its extracts, the chemical structure lends itself to multiple variations. There are about 120 cannabinoids found in cannabis, and several of these other cannabis-derived compounds are discussed in the prior art. The primarily relevant cannabinoids include tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), and cannabicitran (CBT). However, CBD and THC are the compounds that are the focus of most cannabinoid products.

The art related to orally dissolvable tablets includes tablets designed to be dissolved on the tongue rather than be swallowed whole, and such tablets use a wide variety of formulations that differ in large part depending on the active ingredients, which often are to be absorbed through the mucous membranes in the mouth. Also known as orally disintegrating tablets or orally dissolving tablets (ODT), ODTs may have a faster onset of medicinal effects than tablets or capsules. Absorption through the cheek allows some drugs to bypass the digestive tract for rapid systemic distribution. A fast disintegration time and a small tablet weight can enhance absorption in the buccal area. ODTs also have the convenience of a tablet that can be taken without water. However, not all ODTs have buccal absorption and many have similar absorption and bioavailability to standard oral dosage forms with the primary route remaining GI absorption. ODTs also serve as an alternative dosage form for patients who experience dysphagia (difficulty in swallowing) or for where compliance is a known issue and therefore an easier dosage form to take ensures that medication is taken.

The first ODTs disintegrated through effervescence rather than dissolution, and were designed to make taking vitamins more pleasant for children. This method was adapted to pharmaceutical use with the invention of microparticles containing a drug, which would be released upon effervescence of the tablet and swallowed by the patient. Dissolution became more effective than effervescence through improved manufacturing processes and ingredients (such as the addition of mannitol to increase binding and decrease dissolution time). With developments in orally disintegrating tablets, the U.S. Food and Drug Administration (FDA) approved the first ODT drug formulation in 1996.

As described below, embodiments of the present invention include the formulation and formation of edible cannabis in orally dissolvable tablets, including mixtures including a sugar-based solution, such as a maple syrup, using systems and methods different from those of the prior art systems and methods.

### BRIEF SUMMARY OF THE INVENTION

In a first aspect and exemplary embodiment of the invention, a system is provided for mixing and tableting a tablet formulation, in which the system includes a sugar-based solution in a heatable container, a cannabinoid concentrate in a heatable container, a liquid heat bath of, for example, oil, water, or the like for diffused heating of each container, a magnet stirring hot plate for applying bottom heat to a container, and for applying a magnetic force to stir contents of a container, and a magnet stirring pellet for stirring contents of a container. The system also may include an optional radiant heat lamp for applying top radiant heat to a mixture of the sugar-based solution and the cannabinoid concentrate and an optional stirring rod or spatula for manually stirring the mixture as it crystallizes. The system also may include an evaporation oven for desiccating a crystallized mixture made with the aforementioned components, and a pulverization apparatus for pulverizing the desiccated crystallized mixture. The system further may include a mixture of excipients added to the pulverized desiccated crystallized mixture for improving disintegration of ODT tablets including, but not limited to, the FirmaPress LFA tableting mixture. In some embodiments, this mixture includes microcrystalline cellulose, magnesium stearate, silica dioxide, and di-calcium phosphate. Microcrystal line cellulose is an easy flowing powder that adds bulk and weight to a mixture. It offers strong bonding qualities while retaining high flowability, which ensures it is spread evenly throughout a mixture. Magnesium acts as a prime lubricant reducing friction at the point of contact between a tablet surface and the die wall during production, reducing damage to freshly made tablets. It will prevent a formula from sticking to the tableting machine and it is unaffected by process variables (for example, flow, temperature, pressure and level) which occur spontaneously. Magnesium stearate has a high flow rate and is inert. Silicon dioxide absorbs liquid easily, turning liquid ingredients into a free-flowing powder that can be implemented into a formula. It acts as a glidant, which will improve flow qualities but still promotes quick tableting. This ingredient will also reduce trace quantities of moisture which can damage pressing machines over time. It also ensures that your tablets will have uniform content and increases compressibility capabilities.

The system also may include a mixture of tableting additives added to the pulverized desiccated crystallized mixture for improving tableting of ODT tablets. The system likewise may include a tableting machine for tableting ODT formulations into a tablet of an ODT formulation.

In a second aspect and exemplary embodiment of the present invention, a method for combining cannabinoids with a sugar-based solution is provided. This method may include the following steps: creating or providing a sugar-based solution; separately warming the sugar-based solution and a concentrate containing cannabinoids to a first temperature set point; adding the concentrate to the sugar-based solution to create a mixture; applying substantially diffused heat (e.g., from a top and bottom heat source) to the mixture; increasing the temperature of the mixture to a second temperature set point sufficient to initiate crystallization of the mixture; stirring of the mixture at a first stirring speed set point, such as applying a magnetic force to cause a magnetic stirrer to rotate, measured in rotations per minute (RPM); upon the mixture beginning crystallization, raising the temperature to a third temperature set point and raising the RPMs to a second RPM set point; and, upon the mixture reaching full crystallization, stopping heating, the RPMs, and the magnetic force.

In a third aspect and exemplary embodiment of the invention, a method for creating a rapid release, orally dissolvable tablet is provided. This method comprises taking the combined cannabinoids and a sugar-based solution in powder form, mixing with a secondary formulation and/or an excipient mixture, and pressing the resulting mixture into tablet form. In particular, a method for mixing and tableting a formulation is provided, in which the method may include removing heating sources from a crystallized cannabinoid-sugar-based solution mixture and allowing the mixture to cool; desiccating the mixture, such as using an evaporation oven, to remove moisture and reduce moisture-associated stickiness of the mixture; pulverizing the desiccated mixture, such as using a pulverization apparatus, such as including a mortar and pestle; adding a mixture of excipients to the mixture of microcrystalline material for improving disintegration of ODT tablets to be formed from the combined mixture; pulverizing the combined mixture, such as again employing the pulverization apparatus to pulverize the combined mixture; adding a mixture of tableting additives to the combined mixture, or to the microcrystalline material, to improve tableting of ODT tablets to be formed from the resulting combined mixture; pulverizing the combined mixture, such as again employing the pulverization apparatus to pulverize the resulting combined mixture into a tablet formulation; and tableting the formulation into tablets, such as employing a tableting machine for tableting the resulting combined mixture powder into a tablet of an orally disintegrating tablet formulation.

In a fourth aspect and exemplary embodiment of the invention, an orally dissolvable tablet is provided, in which the orally dissolvable tablet comprises a cannabinoid concentrate; and a sugar-based solution; wherein the cannabinoid concentrate and the sugar-based solution form a powder comprising a pulverized crystalline mixture of the cannabinoid concentrate and the sugar-based solution; wherein the powder comprises a tablet formulation; and wherein the tablet comprises the tablet formulation pressed into a tablet form. In further embodiments, the orally dissolvable tablet may further comprise a pulverized mixture of excipients added to the powder comprising the pulverized crystalline mixture; wherein the mixture of excipients is adapted to improve disintegration of the orally dissolvable tablet formed from the powder. In further embodiments, the orally dissolvable tablet may further comprise a pulverized mixture of tableting additives added to the powder comprising the pulverized crystalline mixture; wherein the mixture of tableting additives is adapted to improve tablet formation of the orally dissolvable tablet formed from the powder.

Further aspects of the invention are set forth herein. The details of exemplary embodiments of the invention are set forth in the accompanying drawings and the description below. Other features, objects, and advantages of the invention will be apparent from the description and drawings, and from the claims

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings, in which the same reference numerals denote the same, similar or comparable elements throughout. The exemplary embodiments illustrated in the drawings are not necessarily to scale or to shape and are not to be considered limiting of its scope. For the purpose of illustrating the invention, there is shown in the drawings embodiments which are presently preferred. It should be understood, however, that the invention is not limited to the precise arrangements and instrumentalities shown. In the drawings:

FIG. 1 depicts a system for creating orally dissolvable tablets using a tablet formulation including a pulverized crystalline combination of maple syrup and cannabinoids in accordance with one embodiment of the present invention.

FIG. 2 depicts a flowchart of the steps of creating a crystallized combination of maple syrup and cannabinoids in accordance with one embodiment of the present invention.

FIG. 3 depicts a flowchart of the steps of mixing and tableting a crystallized combination of maple syrup and cannabinoids in accordance with one embodiment of the present invention.

LISTING OF DRAWING REFERENCE NUMERALS
Below are reference numerals denoting the same, similar or comparable elements
throughout the drawings and detailed description of the invention:

| | |
|---|---|
| a system 100 | for mixing and tableting a tablet formulation |
| a maple syrup 102 | in a heatable container |
| a cannabinoid concentrate 104 | in a heatable container |
| a heat bath of oil 106 | for diffused heating of a container |
| a magnet stirring hot plate 108 | for applying bottom heat to a container, and a |
| | magnetic force to stir contents of a container |
| a magnet stirring pellet 110 | for stirring contents of a container |
| a radiant heat lamp 112 | for applying top radiant heat to a mixture |
| a stirring rod or spatula 114 | for manually stirring a mixture |
| an evaporation oven 116 | for desiccating a mixture |
| a pulverization apparatus 118 | for pulverizing a mixture |
| a mixture of excipients 120 | for improving disintegration of ODT tablets |
| a mixture of tableting additives 122 | for improving tableting of ODT tablets |
| a tableting machine 124 | for tableting ODT formulations |
| a tablet 126 | of an ODT formulation |
| a method 200 | of preparing a mixture for tablet formulation |
| warming 202 | separately maple syrup and cannabinoid |
| | concentrate to a first temperature setpoint |
| adding 204 | concentrate to maple syrup |
| applying 206 | a bottom heat source to the mixture |
| applying 208 | a top radiant heat source to the mixture to |
| | raise the mixture to a second temperature |
| | setpoint |
| applying 210 | a magnetic force to cause stirring at a first |
| | RPM setpoint |
| Raising and stirring 212 | the mixture to a third temperature setpoint and |
| | increase stirring to a second RPM setpoint |
| discontinuing 214 | the magnetic force, the stirring, and the |
| | heating |
| a method 300 | for mixing and tableting a formulation |
| removing 302 | heating sources and allowing a mixture to cool |
| desiccating 304 | the mixture to remove moisture, such as using |
| | an evaporation oven |
| pulverizing 306 | the desiccated mixture, using a pulverization |
| | apparatus, e.g., a mortar and pestle |
| adding 308 | a mixture of excipients to the mixture, for |
| | improving disintegration of tablets to be |
| | formed from the combined mixture |
| pulverizing 310 | the combined mixture, e.g., employing the |
| | pulverization apparatus to pulverize the |
| | combined mixture |
| adding 312 | a mixture of tableting additives to the |
| | combined mixture |
| pulverizing 314 | the combined mixture |
| tableting 316 | the formulation into tablets |

### DETAILED DESCRIPTION OF THE INVENTION

Certain terminology may be used in the following description for convenience only and is not limiting. The words “lower” and “upper” and “top” and “bottom” designate directions in the drawings to which reference is made. The terminology includes the words above specifically mentioned, derivatives thereof and words of similar import.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a method” may include a plurality of methods. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

As used herein, all variations of the term “concentrate” may include concentrate of cannabinoids including but not limited to full spectrum, isolate, live rosin, and crystals.

As used herein, all variations of the term “temperature” refer to temperature expressed in degrees Celsius (° C.) or Fahrenheit (° F.), as indicated, unless otherwise specified.

As used herein, “degrees Brix” or “° Bx” is the sugar content of an aqueous solution, and one-degree Brix is 1 gram of sucrose in 100 grams of solution and represents the strength of the solution as percentage by mass. Brix measurements may be made, for example, using a refractometer to determine accurate sugar concentration employing a measurement of the refractive index to determine parameters pertinent for concentration analysis. If the solution contains dissolved solids other than pure sucrose, then the ° Bx only approximates the dissolved solid content.

The present disclosure provides systems and methods for combining cannabinoids with a sugar-based solution, such as a maple syrup. Prior to the combination taking place as described herein, maple syrup is made. Maple syrup has health advantages due to beneficial minerals and nutrients and the high ratio of sucrose to other sugars. For example, maple syrup has been shown to inhibit colorectal cancer cell growth, and is more tolerable to patients having certain conditions, such as cancer, than other sugars such as glucose and fructose. Because darker maple syrup has a higher ratio of sucrose to glucose than lighter maple syrup and has higher antioxidant activity, using darker maple syrup in the invention may offer increased benefits against cancer.

In the present invention, maple syrup may be made by boiling maple sap. The maple sap may be treated with reverse osmosis to increase sugar content before boiling. This may be done with, for example, equipment manufactured by CDL Maple Sugaring Equipment Inc. Boiling may be performed, for example, utilizing a Deluxe evaporator as manufactured by CDL. In one aspect, water content may be evaporated until a first degrees Brix set point is reached at a minimum. In one embodiment of the invention, the first degrees Brix set point is 66.9 degrees Brix.

In another aspect of the invention, air may be injected into the maple syrup while it is boiling. The quantity of air injected into the maple syrup may be controlled via a valve coupled to or integral to the air injection equipment. In one embodiment, the air injection equipment includes a fan with the following specs: bypass motor type; tangential discharge; 3 blower stages; 1 speed; open drip proof enclosure; 92 cubic feet per minute (CFM) air flow @ 2-in. Orifice; 134.0 Vacuum (H₂0 Sealed); 120 Volts; 60/50 Hz; 1 Phase; 13.8 Max Amps; 403 Max Air Watts; Lug Motor Mounting Type; Universal AC/DC Motor Design; All Angle Motor Mounting Position; A Ins. Class; Ball Bearings; 40° C. ambient temperature; 7.2″ Body Diameter; 7-59/64″ overall height; metal mounting brackets; Intermittent Duty; 700 hour average life; UL Recognized (E47185), CSA Certified (LR31393).

Once the syrup has reached 66.9 degrees Brix, a next step is to draw off and filter the maple syrup. In one embodiment of the invention, this may be done using a filter press, a diatomaceous earth filter, and/or combinations thereof. In embodiments in which a sugar-based solution other than maple syrup is used, the sugar-based solution may be made as desired, such as with flavorings or colorings, with a sugar content comparable to that of the aforementioned maple syrup to facilitate crystallization of a later mixture with the cannabinoid concentrate.

### Exemplary Embodiments of the Invention

Numerous possible embodiments of the tablet formulations, tablet formulation systems, and tablet formulation methods are envisioned. Exemplary embodiments of the invention are described below and depicted in the accompanying drawings.

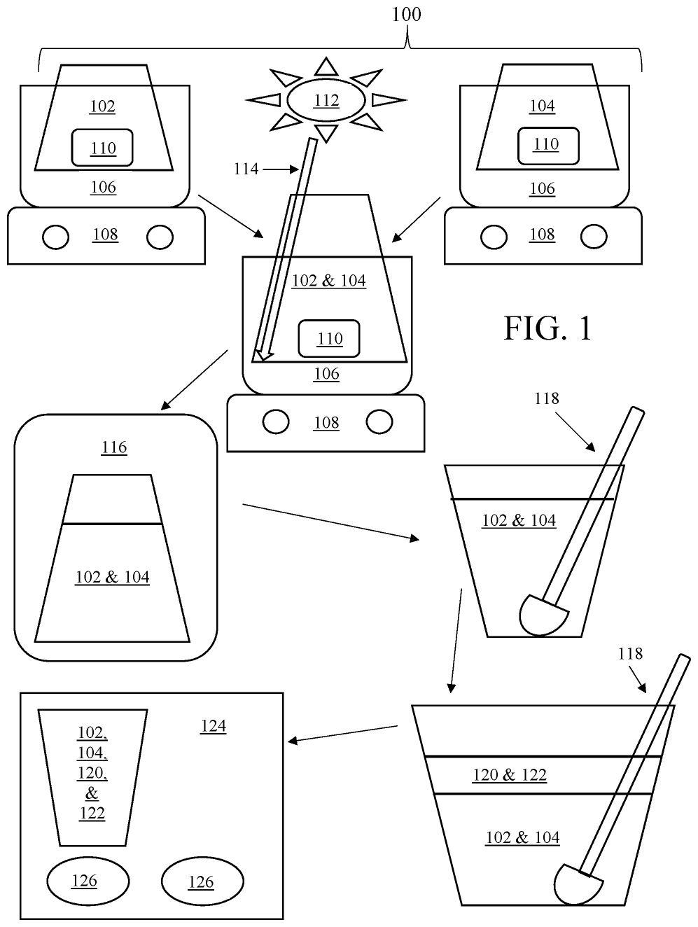

Referring to FIG. 1, FIG. 1 depicts a system 100 for mixing and tableting a formulation in accordance with aspects of the invention. The system 100 for mixing and tableting a tablet formulation may begin with a sugar-based solution, for example, a maple syrup 102 in a heatable container, a cannabinoid concentrate 104 in a heatable container, and a heat bath of oil 106 for diffused heating of each of the containers. The system 100 may employ a magnet stirring hot plate 108 for applying bottom heat to each container and applying a magnetic force to a magnet stirring pellet 110 for stirring contents of a container. The stirring of a mixture may be set to a stirring speed that is measured in rotations per minute (RPM). Once each container has been heated to an initial temperature, the maple syrup 102 and the cannabinoid concentrate 104 may be combined to form a mixture in a container, and mixed while continuing to heat the mixture.

The system 100 again may employ a magnet stirring hot plate 108 for applying bottom heat to the container and applying a magnetic force to a magnet stirring pellet 110 for stirring contents of the container, as well as a radiant heat lamp 112 for applying top radiant heat to the mixture. During the heating process, portions of the mixture will crystallize, and the mixture may be stirred to evenly distribute the crystallization process within the mixture and reduce a risk of overheating portions of the mixture situated in hotter spots in the container. The oil 106 may be heated to about 120° C., for instance, to cause the mixture to boil, while not exceeding 125° C. to avoid burning or caramelizing the mixture. The system 100 also may employ a stirring rod or spatula 114 for manually stirring the mixture, such as may be desirable to vigorously stir the mixture while approaching a mixture temperature of about 120° C. After heating a formulation mixture to a desired point of crystallization, the system 100 may discontinue the heating, magnetic force, and stirring; may remove the magnetic stirring pellet 110; and may remove the container from heating sources 108, 112 to allow the mixture of 102 & 104 to cool somewhat. The system 100 then may use an evaporation oven 116 for desiccating the mixture of polycrystalline material. Desiccation may occur at, for instance, 70° C. for a duration within a range of, for instance, 8-12, 8-18, or 12-18 hours, such as overnight, to remove residual stickiness of the mixture. Further crystallization may occur during the desiccation step.

Once a mixture is sufficiently desiccated and adequately crystallized, the system 100 may use a pulverization apparatus 118 for pulverizing the crystalline mixture into a powder of microcrystalline material. The pulverization apparatus 118 may include a mortar and pestle, as depicted in FIG. 1, for instance. In some embodiments, the system 100 then may add to the mixture of microcrystalline material a mixture of excipients 120 for improving disintegration of ODT tablets to be formed from the combined mixture. The system 100 again may employ the pulverization apparatus 118 to pulverize the combined mixture. In some embodiments, the system 100 also may add to the combined mixture or to the microcrystalline material a mixture of tableting additives 122 for improving tableting of ODT tablets to be formed from the resulting combined mixture. The system 100 again may employ the pulverization apparatus 118 to pulverize the resulting combined mixture. Once the resulting combined mixture is pulverized into a suitable powder, the system 100 may employ a tableting machine 124 for tableting the resulting combined mixture powder into a tablet 126 of an orally disintegrating tablet (ODT) formulation.

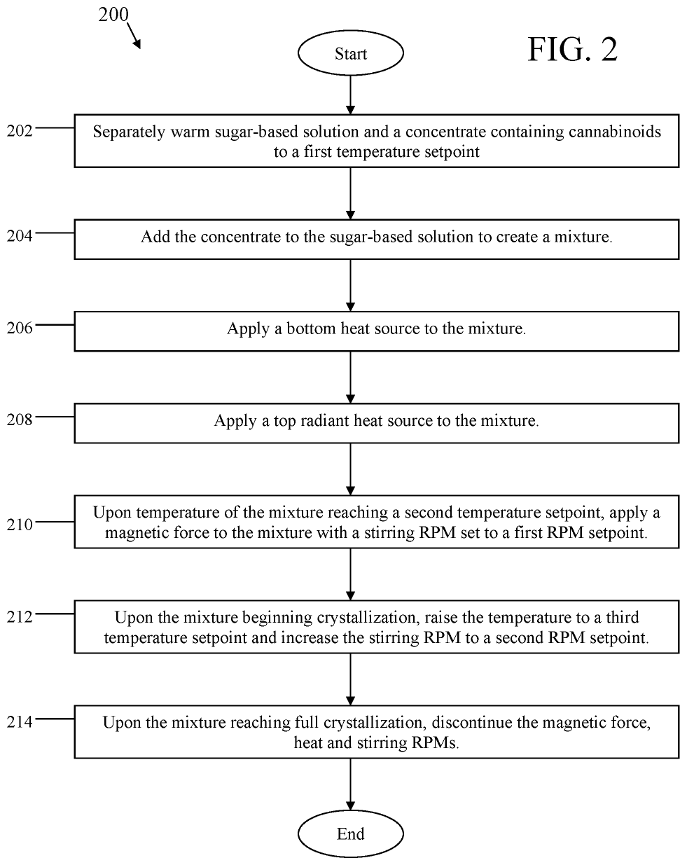

Referring to FIG. 2, FIG. 2 depicts a method 200 for mixing a formulation in accordance with aspects of the invention. One process for creating a combined form of a sugar-based solution, preferably a maple syrup 102 and cannabinoids 104 is as shown in FIG. 2. FIG. 2 depicts the steps of a method 200. The method 200 begins at block 202, at which maple syrup 102 has been created as discussed hereinabove. At step 202, the maple syrup 102 and a concentrate containing cannabinoids 104 are each separately warmed to a first temperature set point. In an embodiment, the concentrate 104 may be a cannabis oil, however the invention is not so limited. At block 204, the method 200 further comprises adding the concentrate 104 to the maple syrup 102 to create a mixture of 102 & 104. In an embodiment, the ratio of maple syrup 102 to the cannabinoid concentrate 104 is 40:1; however, other ratios can be used, depending on factors including, but not limited to, the desired strength of the resulting powder. At block 206, the method 200 further comprises applying a bottom heat source 108 to the mixture. At block 208, the method 200 further comprises applying a top radiant heat source 112 to the mixture. The bottom and top heat sources 108, 112 are applied until the temperature of the mixture reaches a second temperature set point.

At block 210, when the temperature of the mixture of 102 & 104 reaches the second temperature set point, the method 200 further comprises stirring the mixture at a first stirring speed set point, such as applying a magnetic force to the mixture to cause stirring of the mixture using a magnetic stirring pellet 110, measured in rotations per minute (RPM), with an RPM set to a first RPM set point. At block 212, the method 200 further comprises, upon the mixture beginning crystallization, raising the temperature of the mixture to a third temperature set point and increasing the RPMs to a second RPM set point. At block 214, the method 200 further comprises, upon the mixture reaching full crystallization, discontinuing the magnetic force, heat, and RPMs. The method 200 thereafter ends.

Alternate embodiments are envisioned in which a method omits one or more of the steps of heating the mixture or of applying a magnetic force to cause stirring. A combined form of concentrate including cannabinoids 104 and maple syrup 102 may be created according to the following examples.

### Example 1 of Cannabinoid-Maple Syrup Powder

Maple syrup 102 is created utilizing one or more of evaporation, boiling, air injection and/or reverse osmosis. The maple syrup 102 and concentrate 104 are separately warmed to a first temperature set point of 110 degrees Celsius. The concentrate 104 is then added to the maple syrup 102 to create a mixture of 102 & 104. A bottom heat source 108 is then applied to the mixture. In this example, the heat source 108 is at least six hundred (600) joules per second. In an embodiment, a hot plate 108, such as model SH-2 made by Huanghua Faithful Instrument Co., LTD, is used as the bottom heat source 108. In an embodiment, the mixture is stirred while being heated, which helps to evenly distribute the concentrate 104 in the mixture as the crystals begin to form or precipitate. Alternatively, the mixture can be heated at this step without being stirred. Concurrently, a top radiant heat source 112 may be applied. In this embodiment, this top heat source 112 is at least two hundred (200) joules per second. In one embodiment, the top radiant heat source 112 is a Feit Electric 250-watt Incandescent R40 Clear Heat Lamp Reflector 112. However, embodiments are envisioned in which the bottom or top heat source 108, 112 is omitted and/or heat sources of a varying magnitude are utilized.

The temperature of the mixture of 102 & 104 is then raised to a second temperature set point of one hundred and twenty (120) degrees Celsius. Next, a magnetic force is applied to cause stirring using a magnetic stirring pellet 110. In one embodiment, the magnetic force is at least 50 joules per second, and the mixture is stirred at a minimum of six hundred (600) revolutions per minute (RPMs). In an embodiment, a magnetic stirrer hot plate 108 with dual controls and a heating plate stir bar 110 is used, such as model SH-2 made by Huanghua Faithful Instrument Co., LTD. When the mixture starts to crystallize, the temperature is raised to a third temperature setpoint and the revolutions per minute is increased to a second RPM setpoint. In this embodiment, this third temperature setpoint is one hundred and thirty (130) degrees Celsius and the second RPM setpoint is seven hundred (700) RPMs. Once the mixture is fully crystallized, the process is complete.

### Example 2 of Cannabinoid-Maple Syrup Powder

In this example, a greater than or equal to 92% distillate cannabis oil 104 (or other cannabinoid concentrate 104) and the maple syrup 102 are placed in a large beaker at a ratio of 40 mg cannabis oil 104 per 1 mL of maple syrup 102. The beaker in then placed in an oil bath 106 having a temperature of approximately 120° C. until the mixture of 102 &104 comes to a boil. In some embodiments, the mixture is not allowed to exceed 125° C. as such a temperature may cause the mixture to caramelize or burn. As the temperature of the mixture approaches 120° C., the mixture starts to thicken and crystallize, at which point the mixture is stirred vigorously. In an embodiment, the mixture may be stirred with a stirring rod 114 or metal spatula 114. The mixture is then removed from the heat to let the crystals cool. The mixture is stirred occasionally to homogenize the material. Once cool, the crystallized material is placed in a lab oven 116 set to maintain a temperature of 70° C. for a period to remove residual moisture. In some embodiments, this time period may be 8 to 12 hours. In another embodiment, the crystallized material can be freeze dried instead of heated in the evaporation oven 116 in order to remove the residual moisture. Because heat can convert THCA into THC, freeze drying maintains THCA levels, mitigating psychoactive properties. The crystals are then pulverized with, for example, a mortar and pestle 118 into a homogenous powder, and the homogenized powder later is pressed into tablets 126, such as a 700 mg ODT 126. In an exemplary embodiment, the yield is approximately 20 mg CBD per 700 mg tablet 126. Depending on the THC content of cannabis concentrate 104, the 700 mg ODT 126 may have a THC content of about 5 mg, which may be sufficient to cause a psychoactive effect in some people.

### Example 3 of Cannabinoid-Maple Syrup Powder

In this example, a greater than or equal to 92% distillate cannabis oil 104 (or other cannabinoid concentrate 104) and the maple syrup 102 are placed in a large beaker at a ratio of 12 mg of cannabis oil 104 per 1 gram of maple syrup 102 (or 12 grams of cannabis oil 104 per 1 kg of maple syrup 102). The beaker in then placed in an oil bath 106 having a temperature of approximately 120° C. until the mixture comes to a boil. In some embodiments, the mixture is not allowed to exceed 125° C. as such a temperature may cause the mixture to caramelize or burn. As the temperature of the mixture approaches 120° C., the mixture starts to thicken and crystallize, at which point the mixture is stirred vigorously. In an embodiment, the mixture may be stirred with a stirring rod 114 or metal spatula 114. The mixture is then removed from the heat to let the crystals cool. The mixture is stirred occasionally to homogenize the material. Once cool, the crystallized material is placed in a lab oven 116 set to maintain a temperature of 70° C. for a period to remove residual moisture. In some embodiments, this time period may be 8 to 12 hours. In an embodiment, the crystallized material can be freeze dried instead of heated in an evaporation oven 116 in order to remove the residual moisture. The crystals can then be pulverized with, for example, a mortar and pestle 118 into a homogenous powder, and the homogenized powder later is pressed into tablets 126, such as a 700 mg ODT 126. In an exemplary embodiment, the yield is approximately 10 mg CBD per 700 mg tablet 126.

### Alternate Embodiments of Cannabinoid-Maple Syrup Powder

A cannabinoid-maple syrup powder created by one of the processes described above can also be created through alternate means. In an embodiment, the heated mixture of 102 & 104 can be put into a granulated sugar making machine after Step 204 until a granulated mixture is created, after which the granulated mixture can be crushed into a cannabinoid-maple syrup powder. Alternatively, the mixture can be put into a granulated sugar making machine after any of Steps 206, 208, 210 or 212 in order to produce the granulated crystals before they can be pulverized into powder.

In an embodiment, the cannabinoid-maple syrup powder includes cannabinoid contents as follows: 39.4 mg/gram CBD; 6.7 mg/gram THC; 1.9 mg/gram CBC; 0.28 mg/gram CBN; 0.86 mg/gram CBDV; and 0.39 mg/gram CBG. The invention is not limited to this combination of ingredients or this ratio and depends on the initial cannabinoid concentrate 104 mixed with the maple syrup 102. It is envisioned that the composition of the cannabinoid-maple syrup powder can be adjusted to target various conditions. In an embodiment, the powder can be created using a cannabis isolate or full-spectrum extract as an active ingredient. Although the invention described herein utilizes cannabinoid oils, oils that do not contain cannabinoids may be substituted or added, including, without limitation, essential oils, plant-based oils, frankincense oil, myrrh oil, cinnamon oil, eucalyptus oil, or virtually any other non-toxic, bio-compatible oil, including combinations of such oils. Such non-cannabinoid mixtures may be used for many purposes, including, but not limited to, cosmetic facial scrubs, edibles, and other edible products.

Making Tablets with Cannabinoid-Maple Syrup Powder:

The processes described below are alternate steps in creating a rapid release, orally dissolvable tablet 126. The embodiments discussed below form tablets 126 using a maple syrup 102, and other embodiments may use other sugar-based solutions instead. In an embodiment, the cannabinoid-maple syrup powder is combined with a secondary formulation 120 and/or an excipient mixture 120. The combination is homogenized by any means known in the art, including a mortar and pestle 118. In one aspect of the invention, the secondary formulation 120 includes citric acid and sodium bicarbonate, but the invention is not so limited. The sodium bicarbonate may assist binding with the cannabinoid-maple syrup powder, give the tablets 126 a silky feeling and fizzy dissolution, and enhance salivation to aid in the tablet being broken down in the mouth, thereby increasing absorption into the consumer's body. In exemplary embodiments, the citric acid/sodium bicarbonate formulation 120 comprises equal parts citric acid and sodium bicarbonate by weight, by volume, or by moles, depending on the flavor and performance of the final powder composition; however, other ratios may be used. An advantage to using a citric acid is its chemo-protective properties.

In an exemplary embodiment, the secondary formulation 120 may include tableting additives 122, such as magnesium-containing ingredients, including magnesium stearate. Magnesium stearate may be used to reduce the adhesion between the powder (granules) and the punch faces of a tableting machine 124, and thus prevent sticking to tablet punches by offering a non-stick surface. Anti-adherents are also used to help protect tablets 126 from sticking to each other and other surfaces. An exemplary ratio is equal parts citric acid, sodium bicarbonate, and magnesium stearate, although other ratios may be used. The resulting tablet 126 is adapted to be bitten by the consumer and allowed to dissolve. The consumer may then allow saliva to build up and swish it in his or her mouth to aid in the dissolving. The sodium bicarbonate may cause the tablet 126 to be effervescent when placed in a consumer's mouth and subjected to saliva, although this effervescence might be indiscernible to some consumers.

The excipient mixture 120 may include other tableting additives 122 that may be used to increase the capacity of the powder to flow (e.g., anti-adherents) in the tableting machine 124, and allow it to be pressed together more solidly (e.g., binders) into a tablet 126 within a tablet press machine 124. The mixture of tableting additives 122 also may act to increase the hardness, dryness, and/or durability of the resultant tablet 126. In an exemplary embodiment, the excipient mixture 120 includes a mixture of tableting additives 122 that is a combination of microcrystalline cellulose, magnesium stearate, silica (silicon dioxide), and dicalcium phosphate; however, other ingredients creating the same effect may be substituted without departing from the scope hereof.

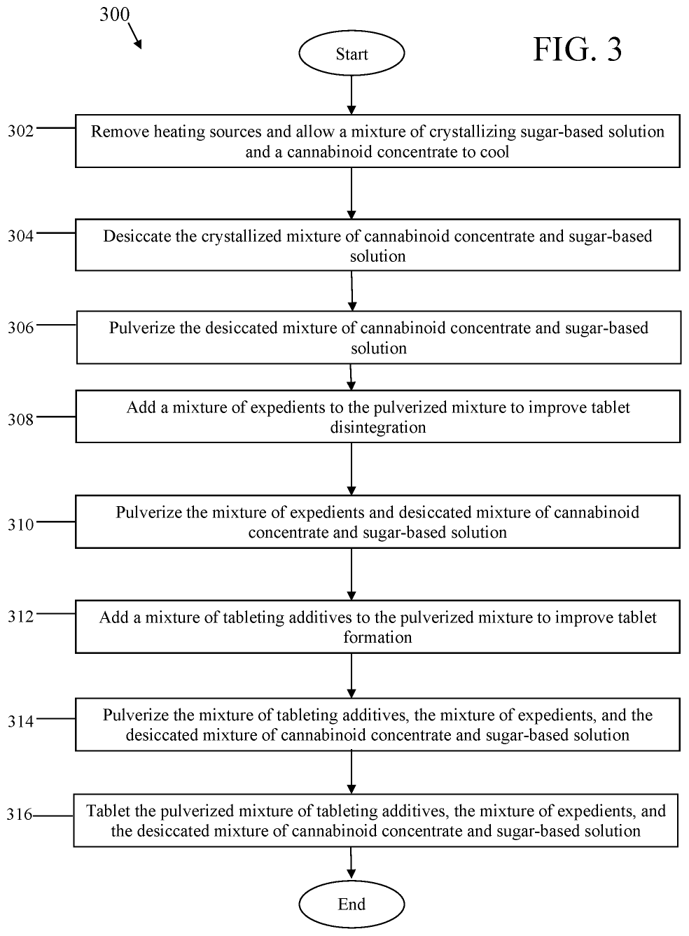

Referring to FIG. 3, FIG. 3 depicts a method 300 for mixing and tableting a formulation in accordance with aspects of the invention. Method 300 may follow a process for creating a combined form of maple syrup 102 and cannabinoids 104 as shown in FIG. 2. FIG. 3 depicts the steps of a method 300. The method 300 begins at block 302, at which point the cannabinoid-maple syrup mixture of 102 and 104 has been created as discussed hereinabove. The step 302 of method 300 may include removing the container from heating sources 108, 112 to allow the mixture of 102 & 104 to cool somewhat. In step 304, the mixture may be desiccated to remove moisture and reduce moisture-associated stickiness of the mixture. Step 304 may use an evaporation oven 116 for desiccating the mixture of polycrystalline material. Desiccation may occur at, for instance, 70° C. for 12-18 hours, such as overnight, to remove residual stickiness of the mixture. Further crystallization may occur during the desiccation step.

Once a mixture is sufficiently desiccated and adequately crystallized, step 306 involves pulverizing the desiccated mixture of 102 & 104. Step 306 may use a pulverization apparatus 118 for pulverizing the crystalline mixture into a powder of microcrystalline material. The pulverization apparatus 118 may include a mortar and pestle 118, as depicted in FIG. 1, for instance. Step 308 then may add to the mixture of microcrystalline material a mixture of excipients 120 for improving disintegration of ODT tablets 126 to be formed from the combined mixture. Following the addition of expedient 120, step 310 pulverizes the combined mixture, and step 310 again may employ the pulverization apparatus 118 to pulverize the combined mixture.

Step 312 then may add a mixture of tableting additives 122 to the combined mixture, or to the microcrystalline material, to improve tableting of ODT tablets 126 to be formed from the resulting combined mixture. Following the addition of tableting additives 122, step 314 pulverizes the combined mixture, and step 314 again may employ the pulverization apparatus 118 to pulverize the resulting combined mixture. Once the resulting combined mixture is pulverized into a suitable powder, step 316 involves tableting the formulation into tablets 126. Step 316 may employ a tableting machine 124 for tableting the resulting combined mixture powder into a tablet 126 of an orally disintegrating tablet (ODT) formulation. Step combinations 308 & 310 and 312 & 314 are optional, and the method 300 may go from step 306 to step 312, from step 306 to step 316, or from step 310 to step 316, depending on the formulation and the decision to include or exclude excipients 120 and/or tableting additives 122.

In an exemplary embodiment, the tablet 126 is approximately 95% cannabinoid-maple syrup powder made utilizing the process described herein for Example 3 above, 2% secondary formulation 120 and 3% excipient tableting mixture 122 by weight. In an exemplary embodiment, the excipient tableting mixture 122 can be omitted, and the combination would be about 95% pulverized cannabinoid-maple syrup powder and 5% secondary formulation 120 by weight. It is envisioned that these ratios could be altered depending on the desired characteristics (e.g., taste, mouth feel, throat feel, after-taste, dosage, dissolution/disintegration rate, or dissolution duration) of the tablets 126. In an exemplary embodiment, the tablets 126 are pressed into a size of 700 mg using a tableting machine 124. In an embodiment, the pills 126 may have a psychoactive effect due to the presence of CBD/THC in a ratio of 23 mg/5 mg per 700 mg tablet 126. As mentioned above, some consumers experience CBD as an antidote to THC that neutralizes the effects of THC, so just as the total amounts of CBD and THC play important roles in dosing, tablet formulation, and tablet formation, the ratio of CBD to THC also may play an important role in dosing, tablet formulation, and tablet formation.

In an exemplary embodiment, a polymer can be used to help bind the cannabinoid-maple syrup powder, however it is not necessary to the invention. Viewed conceptually at a high level, the cannabinoid-maple syrup powder drives the relative strength and consumer-oriented nature of the product (e.g., maple syrup plus cannabinoids at various strengths), whereas the mixture of excipients 120 is directed to the preferred means of consumption and delivery of the product (e.g., tablet 126), while the mixture of tableting additives 122 is directed to the preferred means of forming the delivery means (e.g., tablets 126) that addresses issues arising during formation (e.g., tableting) and after formation (e.g., tablet characteristics like possible stickiness). The excipient mixtures 120 and tableting additives 122 may impact the taste and feel of a tablet 126 by adding acidity, bitterness, fizziness, dryness, hardness, durability, dissolvability, silkiness, and other characteristics, and such characteristics preferably are balanced in the final tablet formulation to achieve an optimal combination that emphasizes favorable characteristics and reduces unfavorable characteristics. Although the tablets may be manufactured with a wide range of excipient mixture by weight, for example, five to twenty-five percent of the total weight, versions having a lower total weight of excipient mixture (e.g., five to ten percent) created a better tasting tablet. Further, the shelf life and storage of the orally dissolvable tablet or other product created by the systems and methods herein should be extended due to the crystalline nature of the product, as this facilitates stabilization and preservation of the active ingredients.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention.

For example, the systems and methods used herein can be incorporated in embodiments other than orally dissolvable tablets including, but not limited to, as a food additive or condiment, pills including swallowed pills, patch applications, inhaled applications, and as an additive to an IV bag for infusion purposes. Also, although the embodiments discussed herein are directed to cannabinoid concentrate, the systems and methods of the present invention may be utilized with pharmaceutical drugs, whether patented or generic, botanicals or the like without departing from the scope hereof.

The foregoing description discloses exemplary embodiments of the invention. While the invention herein disclosed has been described by means of specific embodiments and applications thereof, numerous modifications and variations could be made thereto by those skilled in the art without departing from the scope of the invention set forth in the claims. Modifications of the above disclosed apparatus and methods that fall within the scope of the claimed invention will be readily apparent to those of ordinary skill in the art. Accordingly, other embodiments may fall within the spirit and scope of the claimed invention, as defined by the claims that follow hereafter.

In the description above, numerous specific details are set forth in order to provide a more thorough understanding of embodiments of the invention. It will be apparent, however, to an artisan of ordinary skill that the invention may be practiced without incorporating all aspects of the specific details described herein. Not all possible embodiments of the invention are set forth verbatim herein. A multitude of combinations of aspects of the invention may be formed to create varying embodiments that fall within the scope of the claims hereafter. In addition, specific details well known to those of ordinary skill in the art have not been described in detail so as not to obscure the invention. Readers should note that although examples of the invention are set forth herein, the claims, and the full scope of any equivalents, are what define the metes and bounds of the invention protection.

What is claimed is:

1. A method comprising:
providing a sugar-based solution;
providing a cannabinoid concentrate;
warming the sugar-based solution and the cannabinoid concentrate to a first temperature set point;
mixing the sugar-based solution and the cannabinoid concentrate to create a mixture;
applying heat to the mixture;
increasing the temperature of the mixture to a second temperature set point sufficient to initiate crystallization of the mixture;
stirring the mixture at a first stirring speed set point;
upon the mixture beginning to crystallize, raising the temperature to a third temperature set point and raising the stirring to a second stirring speed set point; and,
upon the mixture reaching sufficient crystallization, stopping the applying of the heat and the stirring.

2. The method of claim 2, wherein:
the warming of the sugar-based solution is performed separately from the warming of the cannabinoid concentrate prior to mixing.

3. The method of claim 2, wherein:
the applying of the substantially diffused heat includes applying heat from a top heat source and a bottom heat source; and
the stirring of the mixture is caused by a magnetic force causing rotation of a magnetic stirrer in the mixture, and the stirring speed is measured in rotations per minute (RPM).

4. The method of claim 2, further comprising:
removing the mixture from each heating source;
allowing the mixture to cool and to further crystallize.

5. The method of claim 4, further comprising:
desiccating the mixture; and
pulverizing the desiccated crystalline mixture into a powder of microcrystalline material.

6. The method of claim 5, wherein:
the desiccating the mixture includes using an evaporation oven to remove moisture and reduce moisture-associated stickiness of the mixture.

7. The method of claim 6, wherein:
the desiccating occurs at approximately 70° C. for a duration within a range of 8-18 hours, during which the mixture further crystallizes.

8. The method of claim 5, further comprising:
adding at least one of a mixture of excipients and a mixture of tableting additives to the pulverized mixture of microcrystalline material; and
pulverizing the at least one of the mixture of excipients and a mixture of tableting additives and the desiccated crystalline mixture into a powder of microcrystalline material.

9. The method of claim 9, further comprising:
tableting the powder of microcrystalline material into the orally dissolvable tablet.

10. The method of claim 1, wherein the heat is substantially diffused.

11. The method of claim 1, wherein the sugar-based solution is maple syrup.

12. A method comprising:
providing a sugar-based solution;
providing a cannabinoid concentrate;
mixing the sugar-based solution and the cannabinoid concentrate to create a mixture;
warming the mixture to a first temperature set point, the first temperature set point sufficient to initiate thickening or crystallization of the mixture;
stirring the mixture at a first stirring speed set point during the thickening or crystallization;
upon the mixture reaching sufficient crystallization, stopping the applying of the heat and the stirring to allow cooling of the mixture;
occasionally stirring the mixture during the cooling of the mixture.

13. The method of claim 12, further comprising:
warming the sugar-based solution to a second setpoint prior to mixing the sugar-based solution and the cannabinoid concentrate to create the mixture; and
warming the cannabinoid concentrate to the second setpoint prior to mixing the sugar-based solution and the cannabinoid concentrate to create the mixture.

14. The method of claim 12, wherein:
the warming of the mixture is caused by a liquid bath; and
the stirring of the mixture is caused by a stirring rod or spatula.

15. The method of claim 12, further comprising:
desiccating the mixture; and
pulverizing the desiccated crystalline mixture into a powder of microcrystalline material.

16. The method of claim 15, wherein:
the desiccating the mixture includes using an evaporation oven to remove moisture.

17. The method of claim 16, wherein:
the desiccating occurs at approximately 70° C. for a duration within a range of 8-18 hours, during which the mixture further crystallizes.

18. The method of claim 15, further comprising:
adding at least one of a mixture of excipients and a mixture of tableting additives to the pulverized mixture of microcrystalline material; and
pulverizing the at least one of the mixture of excipients and the mixture of tableting additives and the desiccated crystalline mixture into a powder of microcrystalline material.

19. The method of claim 19, further comprising:
tableting the powder of microcrystalline material into the orally dissolvable tablet.

20. The method of claim 12, wherein the sugar-based solution is maple syrup.

21. The method of claim 12, wherein the mixture includes 12 grams of cannabinoid concentrate per one kilogram of sugar-based solution.

22. The method of claim 20,
wherein the mixture is approximately ninety-five percent of the total weight of the orally dissolvable tablet;
wherein the mixture of excipients is approximately three percent of the total weight of the orally dissolvable tablet; and
wherein the mixture of tableting additives is approximately two percent of the total weight of the orally dissolvable tablet.

23. The method of claim 18,
wherein the mixture includes approximately 12 grams of cannabinoid concentrate per one kilogram of sugar-based solution;
wherein the mixture of tableting additives includes citric acid and bicarbonate; and
wherein the mixture of excipients includes microcrystalline cellulose, magnesium stearate, silica dioxide, and di-calcium phosphate.
